(19) United States
(12) Patent Application Publication
(10) Pub. No.: US 2024/0233904 A1
(43) Pub. Date: Jul. 11, 2024

(54) HEALTH ADHERENCE SYSTEM WITH LABELING

(71) Applicant: ENDURANCE UNLIMITED INC., Canmore, AB (CA)

(72) Inventors: Peter RAWLEK, Canmore (CA); Kerri DE ZUTTER, Red Deer (CA); Bryan POETZ, Hamilton (CA)

(21) Appl. No.: 18/266,454

(22) PCT Filed: Dec. 13, 2021
(86) PCT No.: PCT/IB2021/000858

§ 371 Date: Nov. 13, 2023

Related U.S. Application Data
(60) Provisional application No. 63/124,466, filed on Dec. 11, 2020.

(51) Int. Cl. G16H 20/30; G16H 15/00; G16H 20/10; G16H 20/60; G16H 20/70; G16H 80/00

(52) U.S. Cl. CPC G16H 20/30; G16H 15/00; G16H 20/10; G16H 20/60; G16H 20/70; G16H 80/00

(57) ABSTRACT

Systems and methods for providing health adherence and related information are disclosed. In an aspect of this disclosure, the system receives a compliance signal from a trainee indicating a status of a trainee's participation in a scheduled health event. A scheduled health event can include a scheduled activity event or a mental health event. The trainee may be assigned to a group having a group label within the system. Information about the trainee within the group may be displayed in response a selection of the group label for the group to which the trainee is assigned.

### CROSS-REFERENCE TO RELATED APPLICATIONS

This application claims priority to and the benefit of U.S. Provisional Patent Application No. 63/124,466, filed Dec. 11, 2020 and entitled “HEALTH ADHERENCE SYSTEM,” which is hereby incorporated by reference herein.

### FIELD

The present disclosure generally relates to a system for providing health adherence information, and more particularly provides a system for evaluating such health adherence information.

### BACKGROUND

The health field is constantly developing new technologies for health treatment, prevention, evaluation, and fitness. Oftentimes, individuals may be busy with activities such as work and school, such that they feel they are unable to attain certain fitness goals without assistance from a healthcare professional and/or a fitness professional. Similarly, individuals may have any number of challenges which impact the introduction of a new habit or new behavior into their lives.

Each individual has unique characteristics and health needs, and therefore, healthcare professionals must make decisions taking into consideration the unique needs of each individual, such as, for example, an individual's prior or current illness, an individual's age or weight level, or an individual's mental health status in order to determine the best method for each individual in order to attain the level of fitness desired. Implementing an effective health plan based on a healthcare professional and/or fitness professional's recommendations for each individual can be difficult given, for example, the busy schedules of professionals. Challenges based on varying schedules of individuals, healthcare professionals, and/or fitness professionals can make in-person visits difficult to schedule and keep, and take up valuable time.

### SUMMARY

Systems and methods for providing health adherence and related information are disclosed. In accordance with an aspect of the disclosure, a method is provided. The method involves receiving, by a processor, a compliance signal from a user indicating a status of a scheduled health event, wherein the compliance signal comprises at least one of a confirmation signal and an omission signal, wherein the confirmation signal indicates completion of the scheduled health event by the user, and wherein the omission signal indicates omission of the scheduled health event by the user. The method further involves adding, by the processor, the compliance signal from the user to a user summary associated with the user, wherein the user summary comprises a plurality of past compliance signals, each of which being associated with a past health event. The method further involves, determining, by the processor, an appropriate compliance category in the user summary for the received compliance signal, wherein the compliance category is at least one of a completion category reflecting completed health events by the user and an omission category reflecting omitted health events by the user. The method further involves assigning, by the processor, the received compliance signal to the appropriate compliance category, wherein the received compliance signal is placed in the completion category in response to the received compliance signal comprising the confirmation signal, and wherein the received compliance signal is placed in the omission category in response to the received compliance signal comprising the omission signal. The method further involves determining, by the processor, a compliance rate for the user, wherein the compliance rate comprises a completion percentage the completed health events relative to a total number of health events, wherein the total number of health events is equal to the completed health events plus the omitted health events, wherein the scheduled health event is at least one of a scheduled activity event, a mental health event, and a lifestyle habit commitment.

In embodiments, the method further involves determining, by the processor, a compliance status for the user based on the compliance rate. In embodiments, receiving the compliance signal comprising an omission signal is in response to an absence of the compliance signal from the user for the scheduled health event, and in response, the receiving the compliance signal comprises generating, by the processor, the compliance signal comprising the omission signal. In embodiments, the method further involves displaying, by the processor, the compliance rate associated with the user on a graphical user interface.

In embodiments, the method further comprises assigning, by the processor, the user to a group having a group label. In embodiments, the method further comprises displaying, by the processor, information associated with the user in response to a selection of the group label. The displayed information may comprise the compliance rate for the user.

In another aspect, a system is disclosed. The system includes a processor, and a tangible, non-transitory memory configured to communicate with the processor, the tangible, non-transitory memory having instructions stored thereon that, in response to execution by the processor, cause the processor to perform operations comprising receiving, by a processor, a compliance signal from a user indicating a status of a scheduled health event, wherein the compliance signal comprises at least one of a confirmation signal and an omission signal, wherein the confirmation signal indicates completion of the scheduled health event by the user, and wherein the omission signal indicates omission of the scheduled health event by the user: adding, by the processor, the compliance signal from the user to a user summary associated with the user, wherein the user summary comprises a plurality of past compliance signals, each of which being associated with a past health event: determining, by the processor, an appropriate compliance category in the user summary for the received compliance signal, wherein the compliance category is at least one of a completion category reflecting completed health events by the user and an omission category reflecting omitted health events by the user: assigning, by the processor, the received compliance signal to the appropriate compliance category, wherein the received compliance signal is placed in the completion category in response to the received compliance signal comprising the confirmation signal, and wherein the received compliance signal is placed in the omission category in response to the received compliance signal comprising the omission signal: and determining, by the processor, a compliance rate for the user, wherein the compliance rate comprises a completion percentage of the completed health events relative to a total number of health events, wherein the total number of health events is equal to the completed health events plus the omitted health events, wherein the schedule health event is at least one of a scheduled activity event, a mental health event, and a lifestyle habit commitment.

In embodiments, the operations further include determining, by the processor, a compliance status for the user based on the compliance rate. In embodiments, the operations further include receiving the compliance signal comprising an omission signal is in response to an absence of the compliance signal from the user for the scheduled health event, and in response, the receiving the compliance signal comprises generating, by the processor, the compliance signal comprising the omission signal. In embodiments, the operations further include displaying, by the processor, the compliance rate associated with the user on a graphical user interface.

In embodiments, the operations further comprise assigning, by the processor, the user to a group having a group label. In embodiments, the operations further comprise displaying, by the processor, information associated with the user in response to a selection of the group label. The displayed information may comprise the compliance rate for the user.

The foregoing features and elements may be combined in various combinations without exclusivity, unless expressly indicated herein otherwise. These features and elements as well as the operation of the disclosed embodiments will become more apparent in light of the following description and accompanying drawings.

### BRIEF DESCRIPTION OF THE DRAWINGS

The subject matter of the disclosure is particularly pointed out and distinctly claimed in the concluding portion of the specification. A more complete understanding of the disclosure, however, may best be obtained by referring to the detailed description and claims when considered in connection with the drawing figures.

FIG. 1 depicts an embodiment of a health adherence system.

FIG. 2 depicts an embodiment of a method for analyzing information as part of the health adherence system.

FIG. 3 depicts an embodiment of a dashboard utilized as part of the health adherence system.

FIG. 4A depicts an embodiment of a dashboard utilized as part of the health adherence system.

FIG. 4B depicts an embodiment of a dashboard utilized as part of the health adherence system.

FIG. 5 depicts a screenshot from a mobile application which is utilized as part of the health adherence system, according to an embodiment of the disclosure.

FIG. 6 depicts a screenshot from a mobile application which is utilized as part of the health adherence system, according to an embodiment of the disclosure.

FIG. 7 depicts a screenshot from a mobile application which is utilized as part of the health adherence system, according to an embodiment of the disclosure.

FIG. 8 depicts a screenshot from a mobile application which is utilized as part of the health adherence system, according to an embodiment of the disclosure.

FIG. 9 depicts a screenshot from a professional portal that is associated with the dashboard and accessed by a healthcare professional as part of the health adherence system.

FIG. 10 depicts a screenshot from a professional portal that is associated with the dashboard and accessed by a healthcare professional as part of the health adherence system.

FIG. 11 depicts a screenshot from a professional portal that is associated with the dashboard and accessed by a healthcare professional as part of the health adherence system.

FIG. 12 depicts a screenshot from a professional portal that is associated with the dashboard and accessed by a healthcare professional as part of the health adherence system.

FIG. 13 depicts a screenshot from a professional portal that is associated with the dashboard and accessed by a healthcare professional as part of the health adherence system.

FIG. 14 depicts a screenshot from a professional portal that is associated with the dashboard and accessed by a healthcare professional as part of the health adherence system.

FIG. 15 depicts an embodiment of a dashboard utilized as part of the health adherence system.

FIG. 16 depicts an embodiment of a dashboard utilized as part of the health adherence system.

### DETAILED DESCRIPTION

This disclosure generally relates to a system and related methods for providing health evaluation and information. The detailed description of various embodiments herein makes reference to the accompanying drawing figures, which show certain embodiments by way of illustration. While these embodiments are described in sufficient detail to enable those skilled in the art to practice the disclosure, it should be understood that other embodiments may be realized and that logical changes may be made without departing from the spirit and scope of the disclosure. Thus, the detailed description herein is presented for purposes of illustration only and should not be viewed as being limiting. For example, the steps recited in any of the method descriptions may be executed in any order and are not limited to the order presented. Moreover, any of the functions or steps may be outsourced to or performed by one or more third parties. Furthermore, any reference to singular includes plural embodiments, and any reference to more than one component may include a singular embodiment.

### Definitions and Interpretation

As used herein, the singular forms “a,” “an” and “the” are used interchangeably and intended to include the plural forms as well and fall within each meaning, unless the context clearly indicates otherwise. Also, as used herein, “and/or” refers to and encompasses any and all possible combinations of one or more of the listed items, as well as the lack of combinations when interpreted in the alternative (“or”).

As used herein, the terms “individual,” “subject,” “client,” “trainee,” and “patient,” are used interchangeably.

As used herein, the term “compliance signal” generally refers to an indicator received by a processor comprising at least one of a “confirmation signal” and an “omission signal.” The confirmation signal and/or the omission signal generally indicate whether or not the trainee has completed a scheduled health event.

As used herein, the term “scheduled health event” refers to any measurable task relating to health. Without limiting the foregoing, a scheduled health event includes any physical activity (which is also referred to herein a as a scheduled activity event), or any mental health event, or any lifestyle habit commitment. A scheduled health event may be based on a recommendation from a healthcare professional. Without limiting the foregoing, a scheduled health event includes the number of workouts that a trainee completes during a defined period of time and the number of minutes of exercise that a trainee completes during a defined period of time. As used herein, the term “scheduled health event” may optionally be referred to herein as “health information”.

As used herein, the term “scheduled activity event” refers to any scheduled event that involves a form of physical exercise or activity. Schedules activity events include, but are not limited to, running, swimming, walking, bicycling, rock climbing, lifting weights, gardening, shoveling, and participating in organized sports.

As used herein, the term “mental health event” refers to any event that impacts, directly or indirectly, an individual's mental health. A mental health event may be based on a recommendation from a healthcare professional.

As used herein, the term “lifestyle habit commitment” includes behavior that has any positive outcome on a trainee's health including, but not limited to any nutritional commitments and taking prescribed medication. A lifestyle habit commitment may be based on a recommendation from a healthcare professional.

As used herein, the term “user summary” refers to a compilation of compliance signals, which may be associated with the relative success or failure of a trainee's completion of scheduled health events over a defined period of time.

As used herein, the term “completion percentage” refers to a percentage of completed scheduled health events by a trainee as compared to a total number of scheduled health events that the trainee could have completed.

As used herein, the term “GUI” refers to a graphical user interface in which a user, including a trainee, can interact with electronic devices through icons and other visual graphics representations.

### DESCRIPTION OF ASPECTS AND EMBODIMENTS OF THE DISCLOSURE

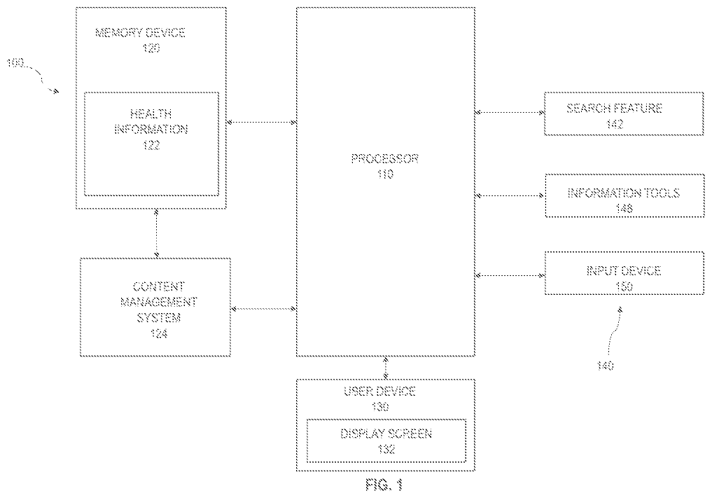

With reference to FIG. 1, an exemplary health evaluation system 100 is disclosed. In embodiments, system 100 allows a healthcare professional to access health-related information easily and efficiently. System 100 can include software integrated with and utilized by a user or trainee's device 130 (e.g., a personal computer, smartphone, tablet, etc.) that has access to a network which allows for other users (e.g., a healthcare professionals) to access and use the information provided by the system 100.

In embodiments, system 100 includes a processor 110, a memory device 120, and a user device 130 that is connected to a network. In embodiments, the user device 130 contains a display screen 132. In embodiments, various features which may be part of a GUI 140 are displayed on display screen 132 of a user device 130. System 100 may be computer-based and have a network interface.

In embodiments, processor 110 may comprise hardware and/or software capable of storing data and/or analyzing information. Processor 110 may comprise a server appliance running a suitable server operating system and having database software installed thereon. Processor 110 may be in electronic communication with memory device 120 and user device 130 and its display screen 132 via a network. Processor 110 may be configured to process information requests, navigate information based on inputs received from a user, and present/display data and information (or cause data and information to be presented and/or displayed) via display screen 132.

In embodiments, memory device 120 may comprise hardware and/or software capable of storing data and/or analyzing information. Memory device 120 may comprise a server appliance running a suitable server operating system and having database software installed thereon. Memory device 120 may be or may comprise a tangible non-transitory computer-readable memory. Instructions stored on the tangible non-transitory memory may allow and/or instruct processor 110 to perform various functions, including navigating and presenting health information, and/or as described herein. Memory device 120 may store health information 122 through which processor 110 may navigate in response to search or navigation inputs received from a user. Health information 122 may be displayed on display screen 132 in response to specific inquiries and selections made by a user.

In embodiments, system 100 may comprise a GUI 140. GUI 140 may take different forms and/or comprise various features, and may be presented on the display screen 132 of user device 130. As a non-limiting example, GUI 140 may comprise a search feature 142, information tools 148, and/or an input device 150. Search feature 142 may include any aspect which allows a user to search for information stored within memory device 120, including but not limited to health information 122. Information tools 148 may include any aspect which allows a user to access components of the system 100 which are designed for providing a user with information. Input device 150 may be any aspect of GUI 140 which allows a user to produce an input signal which may command processor 110 to perform an operation. Serving as a non-limiting example, an input device 150 may be a digital button that is displayed on display screen 132 (e.g., a touch screen) to input certain information.

In embodiments, system 100 is kept up-to-date by new content being added via a content management system 124. In various embodiments, content management system 124 may comprise hardware and/or software capable of receiving and/or storing data, and/or analyzing information. In embodiments, a content management system 124 may be in communication with the memory device 120 and/or processor 110.

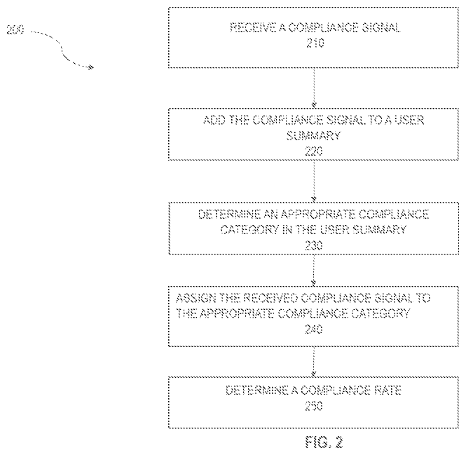

With reference to FIG. 2, an exemplary method 200 is disclosed. In embodiments, the method 200 involves receiving a compliance signal from a trainee that indicates the status of a scheduled health event (step 210). In embodiments, the method 200 further involves adding the compliance signal to a user summary (step 220). In embodiments, the method 200 further involves determining an appropriate compliance category in the user summary for the received compliance signal (step 230). In embodiments, the method 200 further involves assigning the received compliance signal to the appropriate compliance category (step 240). In embodiments, the method 200 further involves determining a compliance rate for the trainee (step 250).

### Dashboard

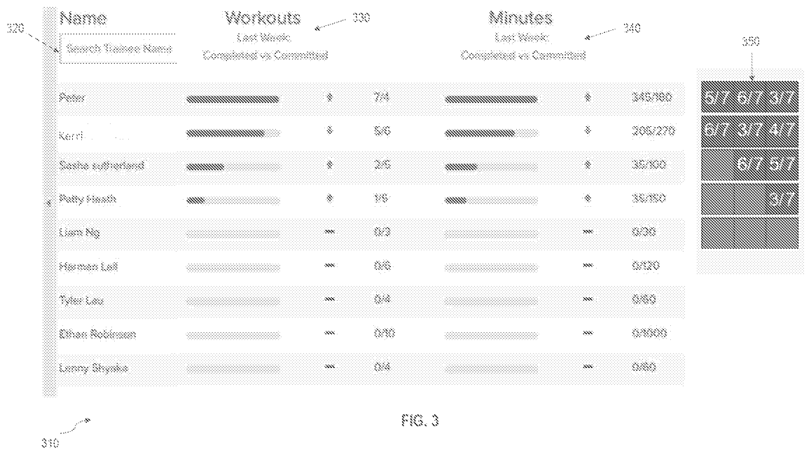

With reference to FIG. 3, an embodiment of a dashboard 310 according to an aspect of the disclosure is provided. In embodiments, the dashboard 310 is displayed on or in association with the processor 110, as referenced in respect of FIG. 1. In practice, a healthcare professional can view the dashboard 310 to determine a trainee's progress or lack thereof. In embodiments, the dashboard 310 is comprised of several components, which can include a name search box 320, a graphical analysis of completed health events 330, and a graphical analysis of the time associated with the completed health events 340.

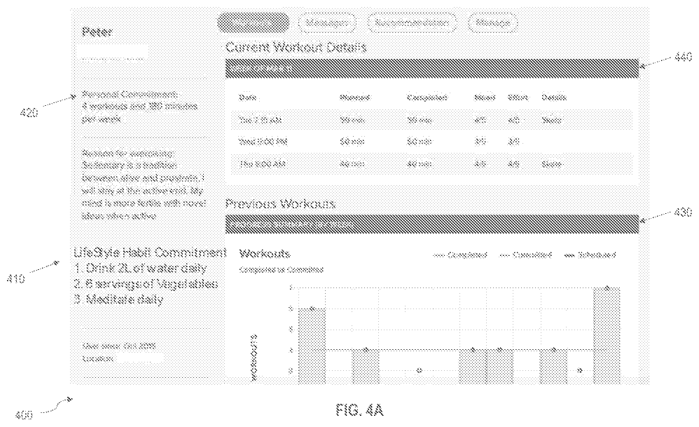

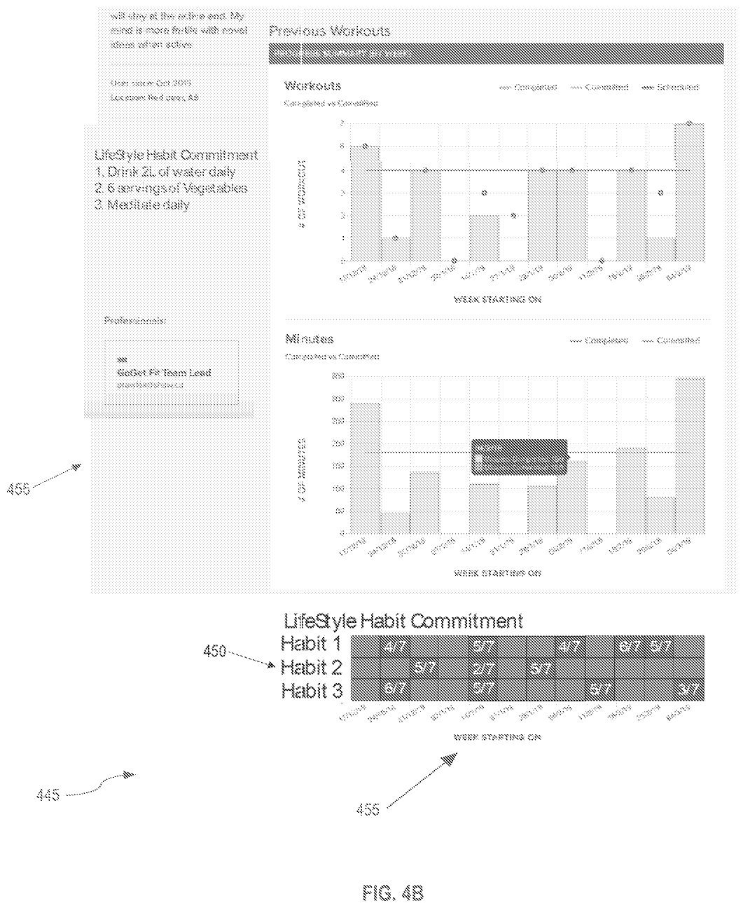

With continued reference to FIG. 3, the dashboard 310 may include a graphical analysis of compliance rates 350. In the example shown in FIG. 3, the compliance rates 350 refer to those associated with certain lifestyle habit commitments, as detailed herein, and as shown, for example, in FIG. 4A.

With continued reference to FIG. 4A, an embodiment of the dashboard 400 as detailed therein includes data concerning lifestyle habit commitments, as shown generally at 410. Further, the dashboard 400 includes scheduled activity events, as shown generally at 420. Elements 410 and 420 reflect the personal commitments that a trainee makes when using the system as described herein. Further, the dashboard 400 contains further numeric analysis 440 and a graphical analysis of scheduled activity events, as shown generally at 430. Element 440 shows an immediate week “live” analysis that represents the “live” completed workouts relative to those scheduled. Element 430 shows a graphical historical compliance success over the previous three (3) months. The foregoing information, while not limited to that as disclosed in FIG. 4A, provides a user of the dashboard 400 with valuable information of a trainee's progress or lack thereof with respect to both lifestyle habit commitments 410 and scheduled activity events 420, as detailed herein in respect of the person commitments being made by a trainee when using the system as described herein.

With reference to FIG. 4B, an embodiment of the dashboard 445 as detailed therein includes data concerning lifestyle habit commitments, as shown generally at 450. Further, the dashboard includes scheduled exercise events, as shown generally at 420. Even more specifically, the dashboard 445 contains a graphical analysis of scheduled activity events, as shown generally at 455. This information, while not limited to that as disclosed in FIG. 4B, provides a user of the dashboard 445 with valuable information of a trainee's progress or lack thereof with respect to both lifestyle habit commitments 450 and scheduled activity events 455. Using the dashboard 445, which provides a platform for selecting and carrying out lifestyle habit commitments 450 and scheduled activity events 455, provides a trainee with an opportunity to establish a pattern of participating in beneficial in lifestyle habit commitments and activity exercise events.

### Mobile Application

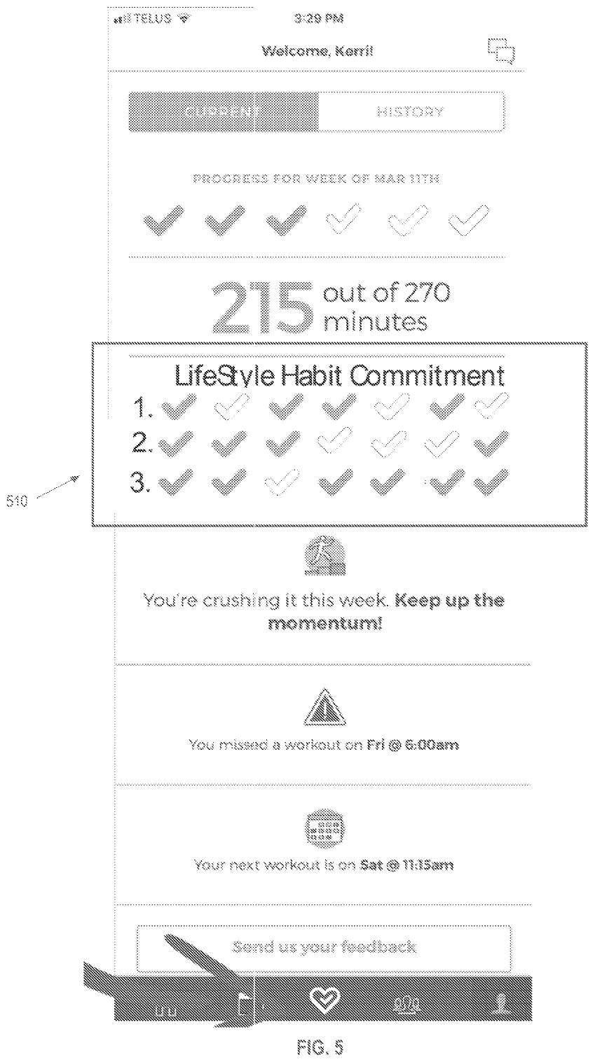

In embodiments, the above-described dashboard (310, 400, 445) interfaces with a trainee's mobile application wherein a trainee inputs a compliance signal, as depicted generally in FIG. 2, and as described herein. More specifically, and as shown generally in FIG. 5, a trainee can input and keep track of both lifestyle habit commitments (as shown generally at 510) and scheduled activity events using their mobile application.

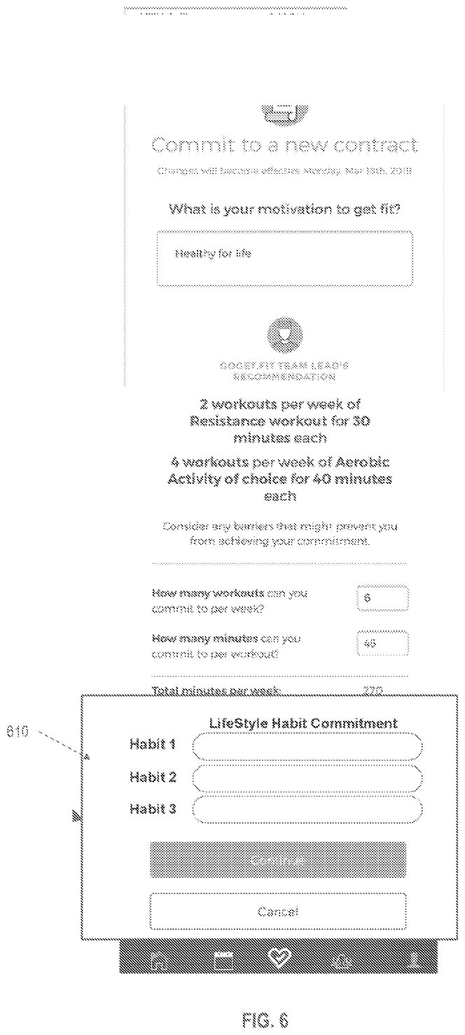

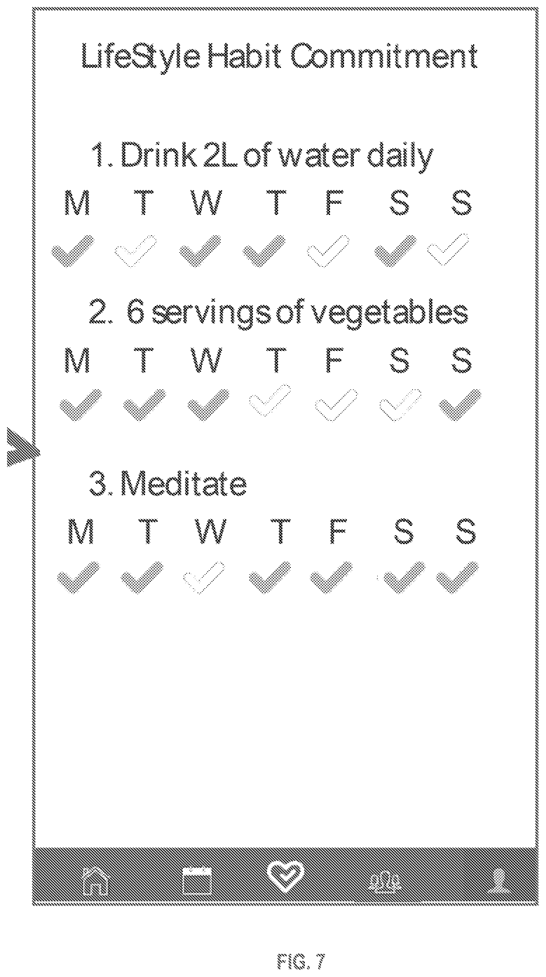

In further embodiments, as shown in FIG. 6, the trainee can introduce trainee-specific lifestyle habit commitments as shown at 610. This is advantageous as it provides a trainee with the ability to introduce a trainee's own (i.e., customized) lifestyle habit commitment and to update and adapt such lifestyle habit commitment over time. Further embodiments are depicted herein in FIG. 7, wherein specific non-limiting examples of lifestyle habit commitments are provided in relation to drinking water, eating vegetables, and meditating.

### Fitness Health Partner Location Feature and Professional Recommendations

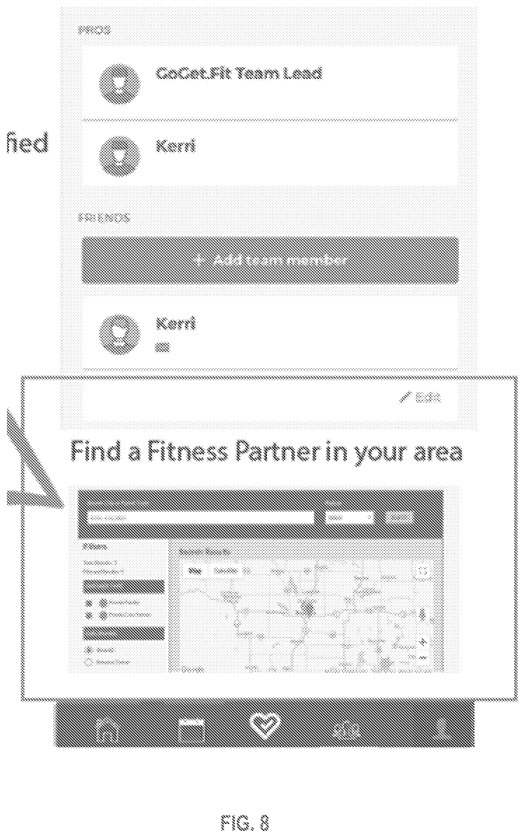

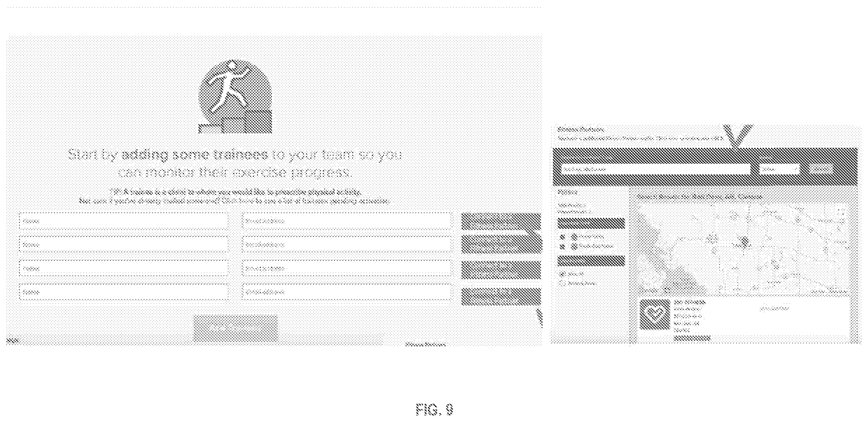

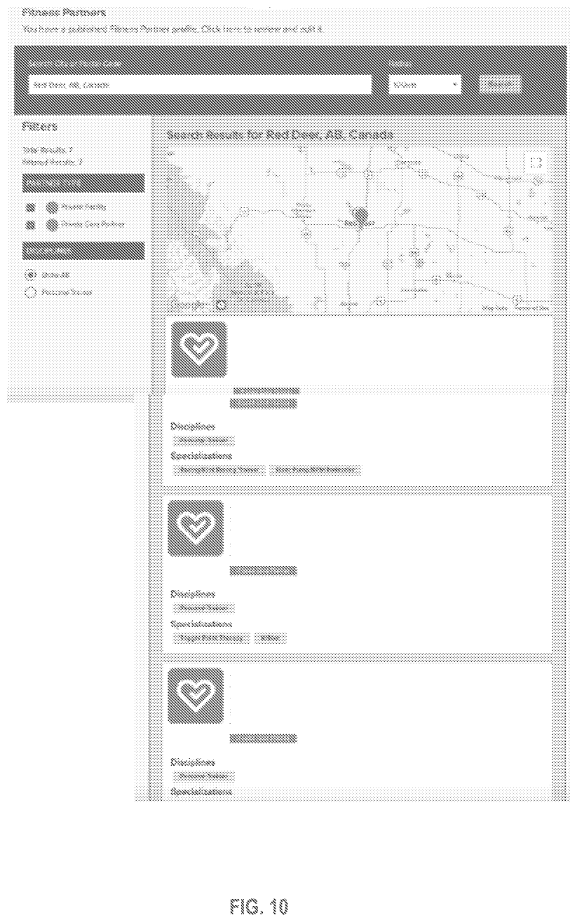

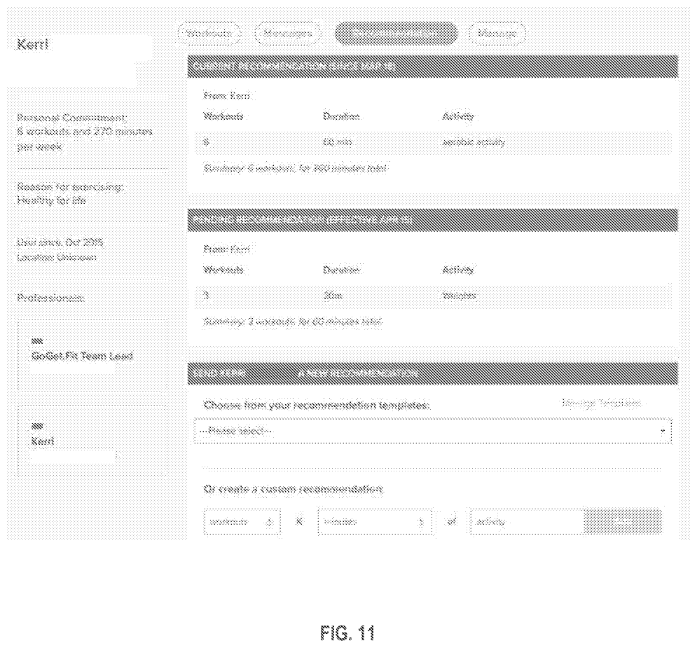

Another feature of the mobile application, as shown generally in FIG. 8 is to provide the trainee with information concerning a fitness/health partner in the trainee's preferred geographic area. A benefit of this feature is that it allows a trainee to identify conveniently-located fitness/health partners which play an integral role in the health adherence system outlined herein. A corollary of this feature is that a professional portal used by a healthcare professional can identify a fitness/health partner for a given trainee, as shown generally in FIG. 9, or in respect of a given geographic location, as shown generally in FIG. 10. Further, the professional portal used by a healthcare professional can contain a feature by which a healthcare professional can send professional recommendations to the trainee, as shown generally in FIG. 11. The professional recommendations are subsequently received by the trainee through the trainee's mobile application.

### Engagement Levels and Defined Group Messaging

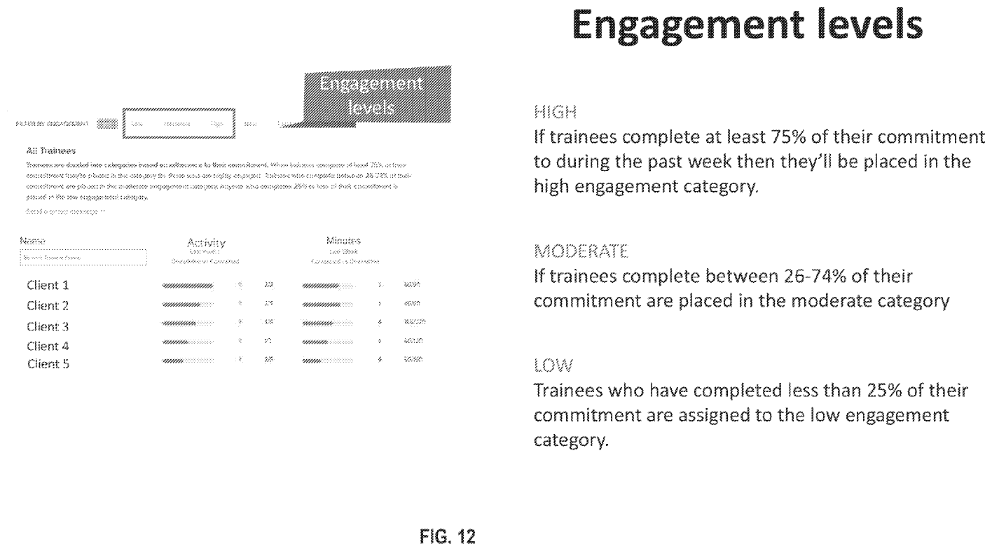

Further, and with reference to FIG. 12, in certain aspects, the system can be modified to define particular engagement levels with respect to a given scheduled health event. For example, and as shown in FIG. 12, which depicts the professional portal used by a healthcare professional, low, medium/moderate, and high engagement levels are pre-established with respect to a particular commitment to a scheduled health event. As depicted in FIG. 12, a high engagement level is consistent with a trainee demonstrating at least a 75% completion of a particular commitment to a scheduled health event. As depicted in FIG. 12, a moderate engagement level is consistent with a trainee demonstrating between about 26%-74% completion of a particular commitment to a scheduled health event. Also, as depicted in FIG. 12, a low engagement level is consistent with a trainee demonstrating less than 24% completion of a particular commitment to a scheduled health event. It is understood that the engagement levels can be pre-determined based on the particular scheduled health event that is being considered.

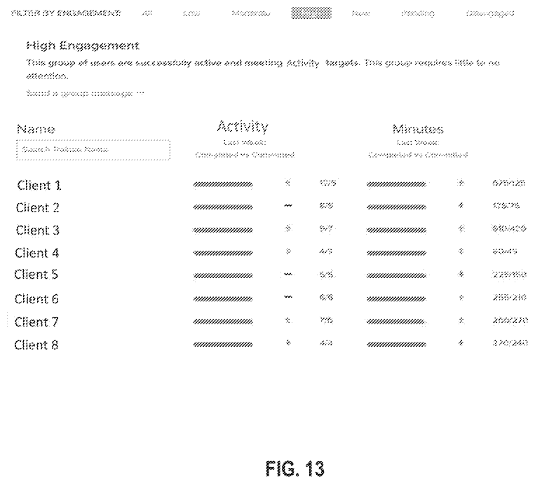

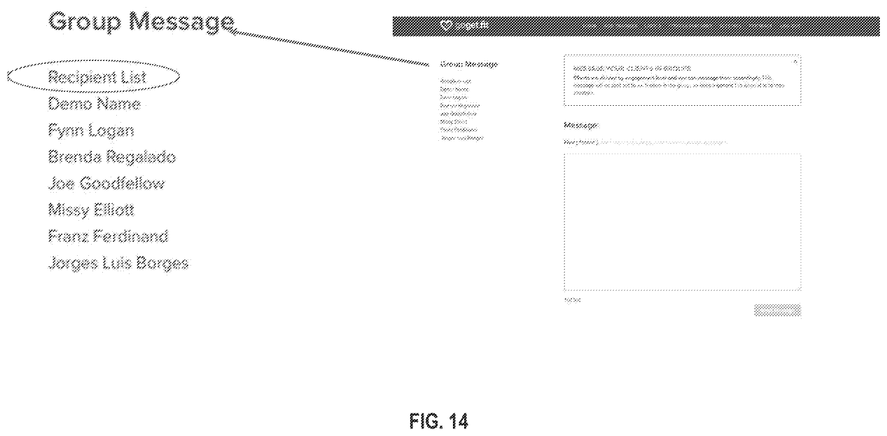

The individuals that demonstrate a particular engagement level can be identified by their engagement level. For example, as shown in FIG. 13, which depicts the professional portal used by a healthcare professional a number of individuals that demonstrated a high engagement level of a particular commitment to a scheduled health event are identified as a cohort or group of individuals. As shown in FIG. 14, which depicts the professional portal used by a healthcare professional the identification of a particular cohort or group allows the healthcare professional to make use of the described system to send a specific message to the entire cohort or group that is consistent with the messaging required for that cohort or group.

### Labels Assigned to Trainee Groups

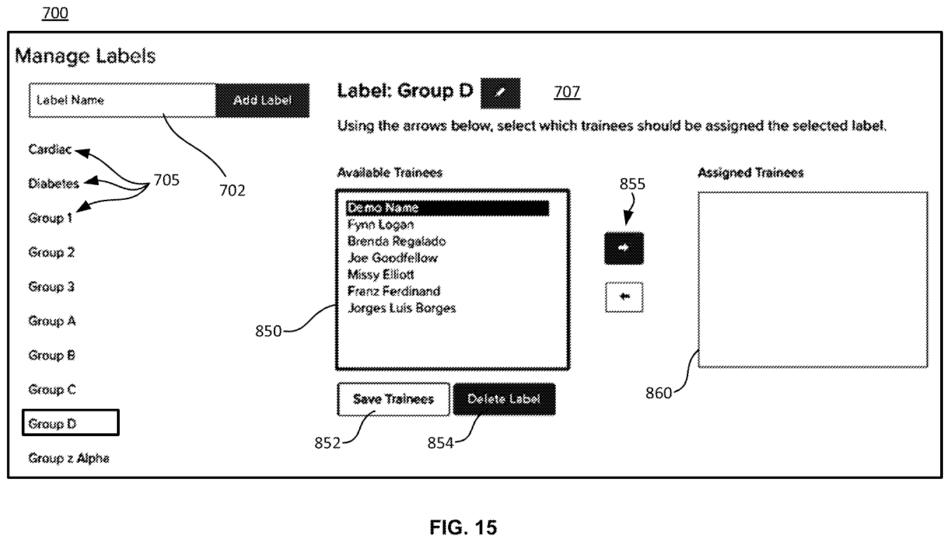

With reference to FIG. 15, in accordance with various embodiments, a label dashboard 700 is depicted. Label dashboard 700 may allow a user (e.g., a healthcare professional) to create new labels for groups of trainees. For example, a user of label dashboard 700 may create a new label by typing the label name into box 702 and adding the label name to the existing labels 705 in a label guide (the label guide may comprise a list of existing labels 705, from which to choose). To view the information associated with a label, one of the existing labels 705 may be selected (e.g., the label Group D may be selected of existing labels 705).

Information associated with the selected group (e.g., Group D) may be displayed in label information display 707 comprised in label dashboard 700. For example, the trainees assigned to Group D may be displayed in the assigned trainees box 860, and trainees available to be assigned to Group D may be displayed in available trainees box 850. To assign a trainee to the selected group, the user may select the trainee's name from available trainees box 850, and select the appropriate transfer arrow 855. In response, the selected trainee's name may be removed from the available trainees box 850 and added to the assigned trainees box 860. In various embodiments, a trainee may be able to be assigned to multiple groups. To remove a trainee from the selected group, the user may select the trainee's name from the assigned trainees box 860, and select the appropriate transfer arrow 855. In response, the selected trainee's name may be removed from the assigned trainees box 860 and added to the available trainees box 850. In response to the desired trainees being assigned to the label and/or selected group, the user may select the save trainees button 852 to save the assignments. Messages (e.g., such as group messages discussed above in relation to FIGS. 12-14) may be sent to the groups under a certain label(s).

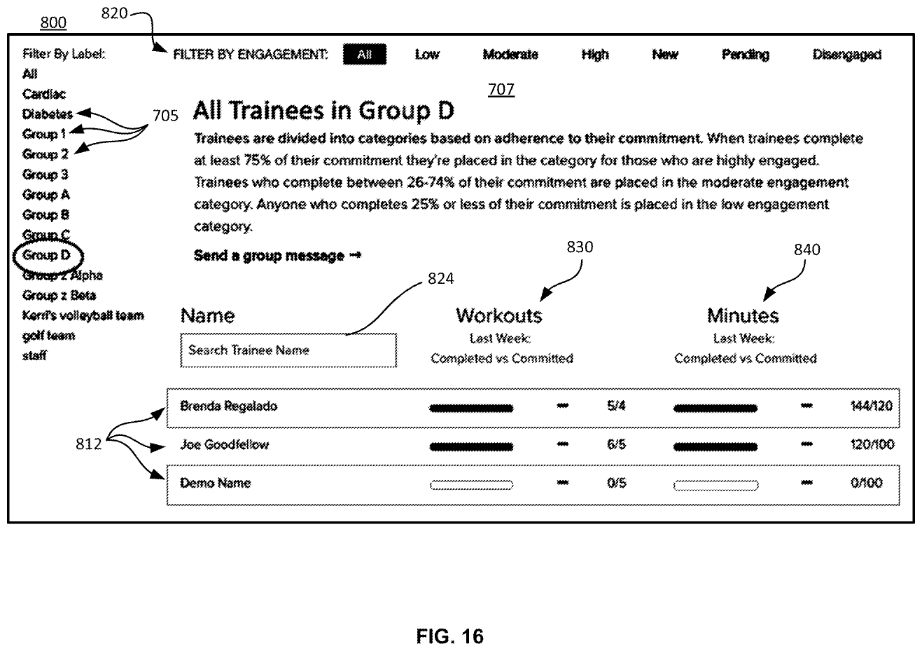

In various embodiments, with reference to FIG. 16, label information display 707 in label dashboard 800 may display information associated with the trainees assigned to the selected label (e.g., Group D). The user may search for a specific trainee assigned to the selected label, for example, by using search box 824. Trainee information associated with the trainees of the selected group may be displayed in any suitable arrangement, such as in trainee rows 812. For example, a trainee row may display the trainee's name, and then information associated with the trainee. As shown in FIG. 16, the engagement levels to the trainee, and/or information related therewith, may be displayed. For example, the trainee Joe Goodfellow may have committed to five health events (e.g., the workouts 830 presented in label information display 707) and 100 minutes associated with the committed health events (e.g., the minutes 840) presented in label information display 707). A graphical representation of the completed health events may be displayed in the associated trainee row 812. For example, for trainee Joe Goodfellow; the graphical representation may show that Joe completed at least 100% of his committed health events (in this case he completed six workouts, which is greater than the five he committed to) and at least 100% of his committed minutes spent in the health events (in this case he completed 120 minutes in his workouts, which is greater than the 100 minutes he committed to).

Label dashboard 800 may further comprise filter options 820 to further allow the user to customize the information presented on label information display 707 regarding a selected label or group. As shown in FIG. 16, filter options 820 allow the user to filter between trainees with different engagement levels (as discussed in relation to FIGS. 12-14), or filter between trainees with other characteristics of interest. In various embodiments, the filter options may offer filtering by any desired information (e.g., the age, gender, weight, health event commitments, etc. of the trainees), including for multiple aspects or characteristics of a trainee.

The following Examples are non-limiting as it relates to this disclosure:

### Example 1: Individual 1 and Scheduled Health Event

Individual 1, also referred to herein as “Peter”, set a scheduled activity event goal of 4 workouts in a given week. Individual 1 also set a scheduled activity event-related number of minutes of workout activity at 180 minutes in a given week. During the given week, Individual 1 completed 7 workouts and 345 minutes of workout activity. This information was introduced into Individual 1's mobile application and was transmitted to the dashboard as shown in FIG. 3.

### Example 2: Individual 2 and Lifestyle Habit Commitment

Individual 2 set pre-determined goals related to certain lifestyle habit commitments. During a given week, out of 7 possible days, Individual 2 drank 2 L of water on 4 of the 7 possible days. During a given week, out of 7 possible days, Individual 2 ate 6 servings of vegetable on 4 of the 7 possible days. During a given week, out of 7 possible days, Individual 2 meditated on 6 of the 7 possible days. This lifestyle habit commitment data is shown in FIG. 7. This lifestyle habit commitment data can be transmitted to a dashboard in accordance with this disclosure.

What is claimed is:

1. A method, comprising:
receiving, by a processor, a compliance signal from a user indicating a status of a scheduled health event, wherein the compliance signal comprises at least one of a confirmation signal and an omission signal, wherein the confirmation signal indicates completion of the scheduled health event by the user, and wherein the omission signal indicates omission of the scheduled health event by the user;
adding, by the processor, the compliance signal from the user to a user summary associated with the user, wherein the user summary comprises a plurality of past compliance signals, each of which being associated with a past health event;
determining, by the processor, an appropriate compliance category in the user summary for the received compliance signal, wherein the compliance category is at least one of a completion category reflecting completed health events by the user and an omission category reflecting omitted health events by the user;
assigning, by the processor, the received compliance signal to the appropriate compliance category, wherein the received compliance signal is placed in the completion category in response to the received compliance signal comprising the confirmation signal, and wherein the received compliance signal is placed in the omission category in response to the received compliance signal comprising the omission signal; and
determining, by the processor, a compliance rate for the user, wherein the compliance rate comprises a completion percentage of the completed health events relative to a total number of health events, wherein the total number of health events is equal to the completed health events plus the omitted health events,
wherein the scheduled health event is at least one of a scheduled activity event, a mental health event, and a lifestyle habit commitment.

2. The method of claim 1, further comprising determining, by the processor, a compliance status for the user based on the compliance rate.

3. The method of claim 1, wherein the receiving the compliance signal comprising an omission signal is in response to an absence of the compliance signal from the user for the scheduled health event, and in response, the receiving the compliance signal comprises generating, by the processor, the compliance signal comprising the omission signal.

4. The method of claim 1, further comprising displaying, by the processor, the compliance rate associated with the user on a graphical user interface.

5. The method of claim 1, further comprising assigning, by the processor, the user to a group having a group label.

6. The method of claim 5, further comprising displaying, by the processor, information associated with the user in response to a selection of the group label.

7. The method of claim 6, wherein the displayed information comprises the compliance rate for the user.

8. A system, comprising:
a processor; and
a tangible, non-transitory memory configured to communicate with the processor, the tangible, non-transitory memory having instructions stored thereon that, in response to execution by the processor, cause the processor to perform operations comprising:
receiving, by the processor, a compliance signal from a user indicating a status of a scheduled health event, wherein the compliance signal comprises at least one of a confirmation signal and an omission signal, wherein the confirmation signal indicates completion of the scheduled health event by the user, and wherein the omission signal indicates omission of the scheduled health event by the user;
adding, by the processor, the compliance signal from the user to a user summary associated with the user, wherein the user summary comprises a plurality of past compliance signals, each of which being associated with a past health event;
determining, by the processor, an appropriate compliance category in the user summary for the received compliance signal, wherein the compliance category is at least one of a completion category reflecting completed health events by the user and an omission category reflecting omitted health events by the user;
assigning, by the processor, the received compliance signal to the appropriate compliance category, wherein the received compliance signal is placed in the completion category in response to the received compliance signal comprising the confirmation signal, and wherein the received compliance signal is placed in the omission category in response to the received compliance signal comprising the omission signal; and
determining, by the processor, a compliance rate for the user, wherein the compliance rate comprises a completion percentage of the completed health events relative to a total number of health events, wherein the total number of health events is equal to the completed health events plus the omitted health events,
wherein the schedule health event is at least one of a scheduled activity event, a mental health event, and a lifestyle habit commitment.

9. The system of claim 8, wherein the operations further comprise determining, by the processor, a compliance status for the user based on the compliance rate.

10. The system of claim 8, wherein the operations further comprise receiving the compliance signal comprising an omission signal is in response to an absence of the compliance signal from the user for the scheduled health event, and in response, the receiving the compliance signal comprises generating, by the processor, the compliance signal comprising the omission signal.

11. The system of claim 8, wherein the operations further comprise displaying, by the processor, the compliance rate associated with the user on a graphical user interface.

12. The system of claim 8, further comprising assigning, by the processor, the user to a group having a group label.

13. The system of claim 12, further comprising displaying, by the processor, information associated with the user in response to a selection of the group label.

14. The system of claim 13, wherein the displayed information comprises the compliance rate for the user.

15. A system, comprising:
a processor; and
a tangible, non-transitory memory configured to communicate with the processor, the tangible, non-transitory memory having instructions stored thereon that, in response to execution by the processor, cause the processor to perform operations comprising:
assigning, by the processor, a user to a group having a group label;
receiving, by the processor, a compliance signal from the user indicating a status of a scheduled health event, wherein the compliance signal comprises at least one of a confirmation signal and an omission signal, wherein the confirmation signal indicates completion of the scheduled health event by the user, and wherein the omission signal indicates omission of the scheduled health event by the user:
adding, by the processor, the compliance signal from the user to a user summary associated with the user, wherein the user summary comprises a plurality of past compliance signals, each of which being associated with a past health event;
determining, by the processor, an appropriate compliance category in the user summary for the received compliance signal, wherein the compliance category is at least one of a completion category reflecting completed health events by the user and an omission category reflecting omitted health events by the user;
assigning, by the processor, the received compliance signal to the appropriate compliance category, wherein the received compliance signal is placed in the completion category in response to the received compliance signal comprising the confirmation signal, and wherein the received compliance signal is placed in the omission category in response to the received compliance signal comprising the omission signal;
determining, by the processor, a compliance rate for the user, wherein the compliance rate comprises a completion percentage of the completed health events relative to a total number of health events, wherein the total number of health events is equal to the completed health events plus the omitted health events; and
displaying, by the processor, the compliance rate for the user in response to a selection of the group label for the group to which the user is assigned.
